(19) United States
(12) Patent Application Publication
(10) Pub. No.: US 2017/0071979 A1
(43) Pub. Date: Mar. 16, 2017

(54) COMPOSITION AND METHOD FOR TREATING OTITIS

(71) Applicant: Veloce BioPharma, LLC, Fort Lauderdale, FL (US)

(72) Inventors: Joseph Capriotti, Christiansted (VG); Kara Capriotti, Fort Washington, PA (US)

(73) Assignee: Veloce BioPharma, LLC, Fort Lauderdale, FL (US)

(21) Appl. No.: 15/341,930

(22) Filed: Nov. 2, 2016

Related U.S. Application Data
(63) Continuation-in-part of application No. 15/213,289, filed on Jul. 18, 2016.
Application No. 15/213,289 is a continuation of application No. 14/116,360, filed on Mar. 18, 2014, now Pat. No. 9,408,867.
(60) Provisional application No. 61/518,709, filed on May 11, 2011.
(60) Provisional application No. 61/518,689, filed on May 11, 2011.
(60) Provisional application No. 61/518,690, filed on May 11, 2011.
(60) Provisional application No. 61/457,699, filed on May 16, 2011.
(60) Provisional application No. 61/627,148, filed on Sep. 19, 2011.
(60) Provisional application No. 61/600,268, filed on Feb. 17, 2012.

(51) Int. Cl. A61K 33/18; A61K 47/48; A61K 9/06; A61K 47/38; A61K 47/10; A61K 47/06; A61K 9/00; A61K 47/20

(52) U.S. Cl. CPC A61K 33/18; A61K 9/0048; A61K 47/48176; A61K 47/20; A61K 47/38; A61K 47/10; A61K 47/06; A61K 9/06

(57) ABSTRACT

The invention concerns a composition and method for treating an otic infection, including otitis externa or otitis media, the composition comprising povidone iodine and dimethylsulfoxide (DMSO) and, optionally, a gelling agent.

### CROSS-REFERENCE TO RELATED APPLICATIONS

This application is a continuation-in-part of pending U.S. patent application Ser. No. 15/213,289, filed Jul. 18, 2016, which is a continuation of U.S. patent application Ser. No. 14/116,360, filed as a 371 application from PCT/US2012/036942 filed May 8, 2012, and issued Aug. 9, 2016 as U.S. Pat. No. 9,408,867, each claiming the benefit of U.S. Provisional Patent Application Nos. 61/518,709, filed May 11, 2011; 61/518,689, filed May 11, 2011; 61/518,690, filed May 11, 2011; 61/457,699, filed May 16, 2011; 61/627,148, filed Sep. 19, 2011; and 61/600,268, filed Feb. 17, 2012. Each of these applications is incorporated herein by reference, in its entirety.

### BACKGROUND

Millions of human patients suffer from infection or inflammation of the ear canal, either the external ear (Otitis externa) or middle ear (Otitis media). For purposes of the subject invention, these conditions may be referred to, collectively, as “otitis.” Otitis can be acute or chronic and may exhibit effusion.

Current treatments for bacterial, fungal/yeast, and viral infections of the ear canal can be ineffective in that they treat only a subset of the causative agent of the infection. Many of the current treatments incorporate undesirable ingredients, such as steroids or other potentially harmful components.

For example, US Patent Applications, Publication Nos. 2009/0263345; 2012/0003174; and 2013/0089510 describe treatment of otitis using PVP-I and a steroid. According to these applications, otitis externa (external ear infection) is an inflammation of the outer ear and ear canal. It is a common cause of earache in humans and a common problem in dogs, cats and other mammals It also occurs in many other species. This disorder involves inflammation of the skin of the ear canal. The inflammation can be caused by active fungal, viral or bacterial organisms. The ear canal skin often swells and may become painful and tender to touch. Otitis media (middle ear infection) occurs in the area between the ear drum and the inner ear, including the Eustachian tube. Otitis media is very common in childhood, with the average toddler experiencing two to three episodes a year, almost always accompanied by a viral upper respiratory infection (URI), mostly the common cold. The rhinoviruses and adenoviruses that cause many common cold symptoms cause swelling and congestion in the inner ear which can permanently damage middle ear structures. Otitis media is also frequently caused by a variety of bacteria and other viruses . Furthermore, ear infection (particularly in children) is one of the many diseases that have become hard to treat with traditional antibiotic drugs because of antibiotic resistant bacteria and antibiotic-resistant microorganisms. Most cases of otitis media, for example, are caused by one of several major pathogens, Streptococcus pneumonia, Haemophilus influenza, Moraxella catarrhalia, Staphylococcus aureus, Staphylococcus epidermidis, or Pseudomonas aeruginosa.

There is thus an urgent need to develop new approaches to prevent and manage these diseases which do not require the use of conventional antibiotics or steroids in view of the known disadvantages of antibiotic resistance, potentially harmful side-effects of steroids, or the like, that may result from the use of compositions that include a conventional antibiotic or steroid.

A recent discovery by Capriotti, et al., has disclosed compositions comprising an iodophor, such as povidone-iodine (PVP-I) as an active ingredient, and dimethyl sulfoxide (DMSO) used as a penetration enhancer for the active ingredient. These compositions were shown to be useful for treating fungal infections of the skin and nails. See, e.g., US Publication No. US2014/0205559 (Capriotti '559), which is incorporated herein by reference in its entirety. PVP-I is a well-known antiseptic with a broad spectrum of activity against viral, fungal, and bacterial species, as well as demodex, and has no known bacterial, fungal or viral resistance. PVP-I has also been shown to inhibit the formation of biofilms and to eliminate biofilms that have already formed.

A variety of organic solvents, including dimethyl sulfoxide (DMSO), are known to enhance the percutaneous absorption of certain medicaments. Nevertheless, it has been a long-accepted in the pharmaceutical arts that DMSO enhances penetration for small molecules or low molecular weight (LMW) compounds or drugs, but does not enhance penetration of high molecular weight (HMW) compounds greater than about 10,000 Daltons, such as polymers.

US Patent Publication Nos. 2009/0263345; 2012/0003174; and 2013/0089510 do not describe steroid-free compositions, and do not recognize or contemplate the advantages of a composition comprising PVP-I and DMSO, as described and claimed herein, for treating otitis.

### BRIEF SUMMARY

A composition of the subject invention can be useful in a method for treating viral infection, fungal or yeast infection, bacterial infection, or amoebal infection or infestation of the ear canal.

A topical composition of the subject invention comprises about 0.1% to about 10% povidone-iodine (PVP-I), and about 30% to about 99% dimethyl sulfoxide (DMSO). A topical gel composition of the subject invention comprises about 0.1% to about 10% povidone-iodine (PVP-I), about 30% to about 99% dimethyl sulfoxide (DMSO), and about 1% to about 10% gelling agent. The topical gel composition of the invention can unexpectedly exhibit greater efficacy in treating otitis, compared to a liquid composition substantially free of a gelling agent and comprising about 0.1% to about 10% povidone-iodine and about 30% to about 99% DMSO. A topical gel composition of the subject invention is unexpectedly highly stable at room temperature in the presence of aqueous or anhydrous ingredients.

A preferred composition comprises about 0.25% to about 0.35%, PVP-I another preferred composition comprises PVP-I at 0.25% to about 0.5%, another composition comprises 1% to about 5% PVP-I. A more preferred composition can comprise about 1% PVP-I to about 2% PVP-I. A PVP-I employing povidone grade of K30 is preferred for use in the subject composition.

A preferred composition comprises about 30% to about 70% DMSO. A more preferred composition can comprise about 40% to about 49% DMSO, and even more preferably, about 44% DMSO.

A preferred composition comprises about 0.5% to about 5% gelling agent. A more preferred composition can comprise about 1-2% gelling agent. A particularly useful composition which has been prepared for testing comprises 2% PVP-I; 44% DMSO; 2% hydroxyethylcellulose; and 52% aqueous solvent. A preferred aqueous solvent is water or isotonic buffer.

A composition of the invention can include a gelling agent, as is well known in the art, which can be selected from a gum, agar, carrageenan, petrolatum, or a cellulosic polymer or the like. One preferred cellulosic polymer as a gelling agent is hydroxyethyl cellulose. An alternative cellulosic polymer gelling agent is hydroxymethyl cellulose.

A composition of the invention preferably comprises povidone-iodine, or PVP-I having an average molecular weight greater than 10,000. More preferably, the composition of the invention comprises PVP-I having an average molecular weight between about 20,000 to about 1,000,000. One preferred embodiment comprises PVP-I having an average molecular weight between about 30,000 to about 60,000, or greater. Each of the PVP-I ingredients is referred to herein as a “high molecular weight PVP-I,” or “HMW PVP-I.”

Another embodiment of a composition according to the subject invention is a stable, topical otic gel formulation comprising about 0.1% to about 10% povidone-iodine (PVP-I); about 30% to about 99% dimethyl sulfoxide (DMSO); and about 1% to about 10% gelling agent; wherein, each ingredient in the composition is otically acceptable, and the otic gel composition exhibits greater efficacy in treating infectious conditions of the ear canal, and particularly the external or middle ear compared to a liquid composition substantially free of a gelling agent and comprising about 0.1% to about 10% povidone-iodine and about 30% to about 99% DMSO.

A preferred otic gel composition comprises about 0.25% to about 2.55% PVP-I. A more preferred otic gel composition comprises about 1% to about 3% PVP-I, and a most preferred otic gel composition comprises about 1% PVP-I.

A composition of the invention preferably comprises povidone-iodine, or PVP-I having an average molecular weight greater than 10,000. More preferably, the composition of the invention comprises PVP-I having an average molecular weight between about 20,000 to about 1,000,000. One preferred embodiment comprises high molecular weight (HMW) PVP-I.

A preferred otic gel composition comprises about 30% to about 70% DMSO. A more preferred otic gel composition can comprise about 40% to about 49% DMSO, and even more preferably, about 44% DMSO.

A preferred otic gel composition comprises about 2% to about 5% gelling agent.

A more preferred otic gel composition can comprise about 4% gelling agent. A particularly useful composition which has been prepared for use in treating infection of the eye or eyelid comprises 1% PVP-I; 44% DMSO; 2% hydroxyethylcellulose; and 53% aqueous solvent. A preferred aqueous solvent is water.

An otic gel composition of the invention can include a gelling agent, as is well known in the art, selected from a gum, agar, carrageenan, petrolatum, or a cellulosic polymer or the like. One preferred cellulosic polymer useful as a gelling agent is hydroxyethyl cellulose (HEC). An alternative cellulosic polymer gelling agent is hydroxymethyl cellulose. In an embodiment, disclosed herein is a steroid-free composition for treating otitis, the composition comprising an iodophor, e.g., povidone-iodine (commonly abbreviated as “PVP-I”) and dimethylsulfoxide (commonly abbreviated as “DMSO”), wherein the composition is capable of penetrating the epithelialized ear canal or is capable of penetrating tympanic membrane to treat the external or middle ear infection.

In an embodiment, a composition disclosed herein for treatment of an otitis is substantially anhydrous. In an embodiment, the composition is anhydrous.

In an embodiment, a composition disclosed herein for treatment of otitis includes PVP-I at about 0.01% to about 10% (w/w). In an embodiment, PVP-I is present in a range selected from the group consisting of about 0.05% to about 10%, about 0.1% to about 5%, about 0.2% to about 2.5%, and about 0.5% to about 1% (w/w). In an embodiment, PVP-I is present in a range selected from the group consisting of about 0.1%, about 0.2%, about 0.3%, about 0.4%, about 0.5%, about 1.0%, about 1.25%, about 1.5%, about 2.0%, about 2.5%, and about 5% (w/w). In an embodiment, PVP-I is present at about 1% (w/w).

In an embodiment, a composition disclosed herein for treatment of otitis further comprises at least one naturopathic substance. In an embodiment, a composition disclosed herein for treatment of otitis further comprises at least one substance selected from the group consisting of Punica Granatum Extract, Camellia Sinensis Leaf Extract, Ascorbic Acid, Calendula Officinalis, Extract, Glycrrhiza Glabra Extract, Allantoin, and Cucumis Sativus Fruit Extract.

In an embodiment, a composition disclosed herein for treatment of otitis further comprises at least one antifungal agent selected from the group consisting of tolnaftate, terbinafine, undecylenic acid, clioquinol, miconazole, miconazole nitrate, clorrinazole, tioconazole, nystatin, terconazoic, butoconazole nitrate, ciclopirox olamine, econazole nitrate, triacetin, flucytosine, haloprogin, and ketoconazole. In an embodiment, an antifungal agent is present in an amount of about 1% to about 25% (w/w).

In an embodiment, disclosed herein is a composition for treating otitis, the composition comprising an iodophor, such as PVP-I, and dimethylsulfoxide (DMSO), wherein the composition is capable of penetrating the epithelialized ear canal or through tympanic membrane to treat the external or middle ear infection, further wherein the composition does not comprise a steroid, or is “steroid-free.”

In an embodiment, the subject composition is formulated for topical administration, as a liquid eardrop formulation, or as a gel. In a preferred embodiment, the subject composition is non-foaming, or is free of a foaming agent.

In an embodiment, a composition of the invention comprises a gelling agent in an amount to form a final composition which is a gel. In one embodiment, the subject composition is formulated as a topical gel.

Each of the embodiments of the composition described herein is free of steroid, i.e., is a steroid-free composition.

In an embodiment, the infection treated is a fungal infection. In an embodiment, the infection is a dermatophyte infection.

In an embodiment, the infection treated is a bacterial ear infection. In an embodiment, the infection is selected from Streptococcal, Staphylococcal, Haemophilus influenza and Pseudomonal infection.

In an embodiment, the infection treated is a viral infection. In an embodiment, the infection is an adenoviral infection.

In an embodiment, the infection treated is an amoebal infection.

In an embodiment, disclosed herein is a method for treating otitis, comprising administering to an infected ear canal a composition disclosed herein, and repeating the contacting step as necessary until the otitis has been treated. In an embodiment, the contacting step is conducted at least once a day. In an embodiment, the contacting step is conducted at least twice a day. In a particularly surprising embodiment, it is found that even a single dose of a highly viscous gel formulation (greater than about 100,000 cps viscosity) was able to completely eradicate co the infection of the middle ear in a child.

In an embodiment, disclosed herein is a method for treating an otitis, comprising administering to an infected ear canal a composition of claim 1 and repeating the administering step for at least one week. In an embodiment, the administering step is repeated for at least two weeks. In an embodiment, the administering step is repeated for at least three weeks. In an embodiment, the administering step is repeated for at least four weeks. In an embodiment, the administering step is repeated for up to 12 weeks.

The subject composition can be useful in treating fungal infection in the ear canal wherein the infection is caused by at least one of the members selected from the group consisting of Trichophyton rubrum, T. mentagrophytes, Epidermophyton floccosum, T. violaceum, Microsporum gypseum, T. tonsurans, T. soudanense, T. verrucosum, and members of Candida spp., Neoscytalidium spp., Scopulariopsis spp., and Aspergillus spp.

### DETAILED DESCRIPTION

The present invention concerns a topical composition, preferably a topical gel composition, comprising an iodophor, a penetration enhancer and, in the case of a gel composition, a gelling agent, wherein the composition is particularly effective in treating viral, demodex, fungal/yeast, bacterial, or amoebal infection that can cause otitis, generally, or Otitis externa or Otitis media, in particular. Thus, the subject invention further comprises a method of treating viral, demodex, fungal/yeast, bacterial, or amoebal infection of the ear canal using a topical composition, preferably a topical gel composition, as disclosed herein. The composition can further comprise optional pharmaceutically acceptable excipients or solvents or co-solvents.

A composition of the subject invention preferably comprises active pharmaceutical ingredient (API) recognized as appropriate and acceptable for use in a pharmaceutical preparation by the United States Food and Drug Administration (FDA). A preferred composition of the invention further comprises inactive ingredients or excipients recognized as appropriate and acceptable for use in a pharmaceutical preparation for topical administration. An FDA-approved API or acceptable inactive ingredient or excipient is referred to herein as “pharmaceutically acceptable.” Accordingly, a topical composition, formulation, or preparation of the subject invention comprising a pharmaceutically acceptable API, inactive ingredient or excipient is referred to herein as a “pharmaceutically acceptable” topical composition.

Similarly, an otic composition of the subject invention comprising FDA-approved active or inactive ingredients acceptable for use in an otic preparation, is referred to herein as a “pharmaceutically acceptable otic composition,” or “optically acceptable” composition, comprising API, excipient, or solvent which is “pharmaceutically acceptable” for otic use, or is “otically acceptable.”

More particularly, the subject invention relates, in a preferred embodiment, to a stable topical composition comprising an iodophor having a molecular weight of greater than 10,000 Daltons, dimethyl sulfoxide (DMSO), and a gelling agent, and optional additional pharmaceutically or otically acceptable excipients or solvents or co-solvents.

The compositions and methods are useful in treating one or any combination of at least two of the above diseases, conditions or pathogens.

Compositions In an embodiment, a composition comprises at least one therapeutic agent and at least one solvent and/or penetrant. In an aspect, an iodophor is a therapeutic agent. In an embodiment, a composition comprises at least one antiseptic compound. In an aspect, an antiseptic compound is a therapeutic agent. In an embodiment, a composition comprises an iodophor antiseptic. “Iodophor”, as used herein, refers to a substance comprising iodine and at least one additional agent (e.g., a solubilizing agent) that releases free iodine when in solution. Examples of iodophors include, but are not limited to, povidone iodine (PVP-I), iodine tincture, Lugol's solutions, and iodine salts (e.g., potassium iodide, sodium iodide).

In an embodiment, a composition comprises at least one iodophor. The compositions encompass any iodophor, as well as iodophors as yet to be developed or discovered. In an embodiment, the iodophor is PVP-I. In another embodiment, a composition comprises iodine. In an embodiment, a composition comprises iodine and at least one iodophor.

In an embodiment, PVP-I functions a therapeutic agent in a composition. In an aspect, a PVP-I therapeutic agent functions as an antiseptic. In another embodiment, PVP-I functions as a preservative in a composition. In an aspect, a PVP-I preservative functions as an antiseptic. In another aspect, a PVP-I preservative functions as a stabilizer. In an embodiment, PVP-I functions in at least once capacity in a composition. In another embodiment, PVP-I functions in at least two capacities in a composition.

In another embodiment, a composition further comprises at least one non-iodophor, non-iodine therapeutic agent. In an embodiment, the at least one non-iodophor, non-iodine therapeutic agent is an antiseptic. In another embodiment, the at least one non-iodophor, non-iodine therapeutic agent is not an antiseptic.

In an embodiment, the at least one non-iodophor, non-iodine therapeutic agent is an antifungal agent. Suitable antifungal agents include, for example, allylamines and azoles. In an embodiment, an antifungal agent includes, but is not limited to, tolnaftate, terbinafine, undecylenic acid, clioquinol, miconazole, miconazole nitrate, clorrinazole, tioconazole, nystatin, terconazoic, butoconazole nitrate, ciclopirox olamine, econazole nitrate, triacetin, flucytosine, haloprogin, and ketoconazole.

In an embodiment, a composition comprises one or more naturopathic substances. Naturopathic substances include, but are not limited to, Punica Granatum (Pomegranate) Extract, Camellia Sinensis Leaf (Green Tea) Extract, Ascorbic Acid (Vitamin-C), Calendula Officinalis Extract, Glycrrhiza Glabra (Licorice) Extract, Allantoin, and Cucumis Sativus (Cucumber) Fruit Extract. In an embodiment, a composition comprises DMSO, PVP-I, Punica Granatum (Pomegranate) Extract, Camellia Sinensis Leaf (Green Tea) Extract, Ascorbic Acid (Vitamin-C), Calendula Officinalis Extract, Glycrrhiza Glabra (Licorice) Extract, Allantoin, and Cucumis Sativus (Cucumber) Fruit Extract.

In an embodiment, a composition comprises at least one solvent and/or penetrant. In an embodiment, a single component may function as both a solvent and a penetrant in the composition. In an embodiment, a composition comprises DMSO. In an aspect, DMSO functions as a penetrant for the active component. In an aspect, DMSO functions as a solvent. In yet another aspect, DMSO functions as both a solvent and a penetrant. In an embodiment, DMSO is the sole penetrant in a composition. In an embodiment, DMSO is the sole solvent in a composition. In an embodiment, DMSO is the sole penetrant and solvent in a composition.

In an embodiment, a composition comprises PVP-I and DMSO. In another embodiment, a composition consists of PVP-I and DMSO. In yet another embodiment, a composition consists essentially of PVP-I and DMSO.

One of skill in the art will understand, based on the disclosure herein, how to identify a penetrant useful for the compositions and methods encompassed herein. In an embodiment, a penetrant is one which is useful for enabling the composition to penetrate the unguis. By way of a non-limiting example, methylsulfonylmethane may be used as a penetrant in a composition as encompassed herein.

In an embodiment, a composition comprises at least one co-solvent. In an embodiment, a composition comprises DMSO as a primary solvent, and further comprises at least one co-solvent. In an embodiment, water is a co-solvent. In an embodiment, a composition comprises DMSO as the primary solvent and water as a co-solvent. In an embodiment, a composition consists of DMSO as the primary solvent and water as the co-solvent. In another embodiment, a composition consists essentially of DMSO as the primary solvent and water as the co-solvent. In an embodiment, a composition comprises at least one co-solvent such as, but not limited to, water, or ethanol. In an embodiment, a co-solvent is one or more polar aprotic solvent. In an embodiment, a co-solvent is ethyl acetate. In an embodiment, a co-solvent is at least one of ethyl acetate, acetone, acetonitrile, tetrahydrofuran, methylene chloride, and dimethyl formamide. One of skill in the art will understand the advantages and limitations of the use of co-solvents, based on the properties and physical effects of such potential co-solvents, in view of the disclosure set forth herein.

In an embodiment, a composition comprises at least one excipient such as, but not limited to, sodium chloride, sodium dihydrogen phosphate monohydrate, disodium hydrogen phosphate anhydrous and water. The compositions encompassed herein will be understood to optionally include one or more other excipients as known to those skilled in the art. One of skill in the art will know how to identify such an excipient as useful in the present compositions and methods, for example, when such an excipient enhances the therapeutic effectiveness, stability, or potency of a composition or method.

Dosages, Forms and Formulations

In an embodiment, a composition comprises a therapeutically effective amount of at least one therapeutic agent. The term “therapeutically effective amount” is used herein, unless otherwise indicated, to describe an amount of a compound which, in context, is used to produce or effect an intended therapeutic result. In an embodiment, the intended therapeutic result relates to the treatment of onychomycosis. In an embodiment, a therapeutically effective amount is that amount which is sufficient to treat an otitis, and the treatment of the otitis includes at least one of preventing or slowing the progression of the infection, preventing the spread of the infection, eradicating at least some of the infection, and eradicating the entire infection. In an aspect, a therapeutically effective amount may be determined based on a single dosage or it may be determined based on multiple dosages of the composition. It will be understood that determination of the therapeutically effective amount may require trial and error, and may require adjustment of the dosage and or dosing regimen. Such therapeutic optimization and adjustment is encompassed by the methods encompassed herein.

The term “pharmaceutically acceptable”, as used herein with respect to a compound or composition, refers to a form of the compound or composition that can increase or enhance the solubility or availability of the compound in a subject, in order to promote or enhance the bioavailability of the compound or composition. In an aspect, the disclosure herein also encompasses pharmaceutically acceptable, hydrates, solvates, stereoisomers, or amorphous solids of the compounds and compositions embodied herein.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 1990, incorporated herein by reference). Except insofar as any conventional carrier is incompatible with the active ingredient or method of use, its use in the pharmaceutical compositions is contemplated.

Percentages set forth herein are (w/w), with respect to the specified component in the overall composition, unless otherwise indicated. For example, a composition comprising 1% PVP-1 and 99% DMSO has 1% PVP-I by weight, with respect to the total composition.

In an embodiment, a composition comprises an iodophor in the range of about 0.01% to about 15%. In another embodiment, a composition comprises an iodophor in the range between 0.05% and 12.5%. In another embodiment, a composition comprises an iodophor in the range between 0.05% and 10.0%. In another embodiment, a composition comprises an iodophor in the range between 0.1% and 10.0%. In another embodiment, a composition comprises an iodophor in the range between 0.1% and 5.0%. In another embodiment, a composition comprises an iodophor in the range between 0.25% and 9.0%. In another embodiment, a composition comprises an iodophor in the range between 0.2% and 2.5%. In another embodiment, a composition comprises an iodophor in the range between 0.5% and 7.5%. %. In another embodiment, a composition comprises an iodophor in the range between 0.5% and 1.0%. In another embodiment, a composition comprises an iodophor in the range between 0.75% and 5.0%, and in yet another embodiment, between 1.0% and 4.0%. In an embodiment, a composition comprises an iodophor in the range of about 0.1% to about 2.5%, about 0.2% to about 2.0%, about 0.3% to about 1.0%, and about 0.4% to about 0.75%.

In an embodiment, a composition comprises elemental iodine in the range of about 0.01% to about 15%. In another embodiment, a composition comprises elemental iodine in the range between 0.05% and 12.5%. In another embodiment, a composition comprises elemental iodine in the range between 0.05% and 10.0%. In another embodiment, a composition comprises elemental iodine in the range between 0.1% and 10.0%. In another embodiment, a composition comprises elemental iodine in the range between 0.1% and 5.0%. In another embodiment, a composition comprises elemental iodine in the range between 0.25% and 9.0%. In another embodiment, a composition comprises elemental iodine in the range between 0.2% and 2.5%. In another embodiment, a composition comprises elemental iodine in the range between 0.5% and 7.5%. %. In another embodiment, a composition comprises elemental iodine in the range between 0.5% and 1.0%. In another embodiment, a composition comprises elemental iodine in the range between 0.75% and 5.0%, and in yet another embodiment, between 1.0% and 4.0%. In an embodiment, a composition comprises elemental iodine in the range of about 0.1% to about 2.5%, about 0.2% to about 2.0%, about 0.3% to about 1.0%, and about 0.4% to about 0.75%.

In an embodiment, a composition comprises PVP-I in the range of about 0.01% to about 15%. In another embodiment, a composition comprises PVP-I in the range between 0.05% and 12.5%. In another embodiment, a composition comprises PVP-I in the range between 0.05% and 10.0%. In another embodiment, a composition comprises PVP-I in the range between 0.1% and 10.0%. In another embodiment, a composition comprises PVP-I in the range between 0.1% and 5.0%. In another embodiment, a composition comprises PVP-I in the range between 0.25% and 9.0%. In another embodiment, a composition comprises PVP-I in the range between 0.2% and 2.5%. In another embodiment, a composition comprises PVP-I in the range between 0.5% and 7.5%. %. In another embodiment, a composition comprises PVP-I in the range between 0.5% and 1.0%. In another embodiment, a composition comprises PVP-I in the range between 0.75% and 5.0%, and in yet another embodiment, between 1.0% and 4.0%. In an embodiment, a composition comprises PVP-I in the range of about 0.1% to about 2.5%, about 0.2% to about 2.0%, about 0.3% to about 1.0%, and about 0.4% to about 0.75%.

In an embodiment, a composition comprises PVP-I at about 0.001%, about 0.005%, about 0.01%, about 0.05%, about 0.1%, about 0.2%, about 0.3%, about 0.4%, about 0.5%, about 0.6%, about 0.7%, about 0.8%, about 0.9%, about 1.0%, about 1.25%, about 1.50%, about 1.75%, about 2.0%, about 2.5%, about 5%, about 7.5%, about 10%, about 12.5, or about 15.0%. In an embodiment, a composition comprises PVP-I at 0.001%, 0.005%, 0.01%, 0.05%, 0.1%, 0.2%, 0.3%, 0.4%, 0.5%, 0.6%, 0.7%, 0.8%, 0.9%, 1.0%, 1.25%, 1.5%, 1.75%, 2.0%, 2.25%, 2.5%, 5%, 7.5%, 10.0%, 12.5%, or 15%. In another embodiment, a composition comprises PVP-I at about 1%, about 2%, about 3%, about 4%, about 5%, about 6%, about 7%, about 8%, about 9%, about 10%. In another embodiment, a composition comprises PVP-I at about 0.1% or less, about 0.5% or less, about 1% or less, about 2% or less, about 3% or less, about 4% or less, about 5% or less, about 6% or less, about 7% or less, about 8% or less, about 9% or less or about 10% or less. In another embodiment, a composition comprises PVP-I at about 0.01% or more, about 0.05% or more, about 0.075% or more, about 0.1% or more, about 0.2% or more, about 0.3% or more, about 0.4% or more, about 0.5% or more, about 0.6% or more, about 0.7% or more, about 0.8% or more, about 0.9% or more, about 1% or more, about 2% or more, about 3% or more, about 4% or more, about 5% or more, about 6% or more, about 7% or more, about 8% or more, about 9% or more or about 10% or more. In another embodiment, a composition comprises PVP-I at 0.001%, 0.005%, 0.01%, 0.05%, 0.1%, 0.2%, 0.3%, 0.4%, 0.5%, 0.6%, 0.7%, 0.8%, 0.9%, 1.0%, 2.0%, 3.0%, 4.0%, 5.0%, 6.0%, 7.0%, 8.0%, 9.0% or 10.0%.

In an embodiment, a composition comprises DMSO and PVP-I. In an embodiment, a composition consists essentially of DMSO and PVP-I. In an embodiment, a composition consists of DMSO and PVP-I. In an embodiment, a composition is anhydrous. In an embodiment, a composition is substantially anhydrous. In an embodiment, a composition comprises a measurable amount of water.

In an embodiment, anhydrous DMSO is used in a composition. In an embodiment, substantially anhydrous DMSO is used in a composition. It will be understood by one of skill in the art that DMSO can be produced and/or obtained in differing grades, and that one of the variables among DMSO preparations of different grades is the water content. By way of example, DMSO may be completely anhydrous (also referred to herein simply as “anhydrous”), substantially anhydrous, or may contain water to a measurable degree. It will be understood that the amount of measurable water in a DMSO preparation may vary based on limitations of the instrumentation and techniques used to make such measurements. In an embodiment, DMSO that is not completely anhydrous may be substantially anhydrous and contain water at a level below levels of detectability. In an embodiment, DMSO that is not completely anhydrous may contain water, wherein the water content is about at least 0.01%, about at least 0.02%, about at least 0.03%, about at least 0.04%, about at least 0.05%, about at least 0.06%, about at least 0.07%, about at least 0.08%, about at least 0.09%, about at least 0.1%, about at least 0.2%, about at least 0.3%, about at least 0.4%, about at least 0.5%, about at least 0.6%, about at least 0.7%, about at least 0.8%, about at least 0.9%, about at least 1.0%, about at least 1.5%, about at least 2.0%, about at least 2.5%, about at least 5%, about at least 7.5%, about at least 10%, about at least 12.5%, or greater. In an embodiment, DMSO that is not completely anhydrous may contain water, wherein the water content is about less than 0.01%, about less than 0.02%, about less than 0.03%, about less than 0.04%, about less than 0.05%, about less than 0.06%, about less than 0.07%, about less than 0.08%, about less than 0.09%, about less than 0.1%, about less than 0.2%, about less than 0.3%, about less than 0.4%, about less than 0.5%, about less than 0.6%, about less than 0.7%, about less than 0.8%, about less than 0.9%, about less than 1.0%, about less than 1.5%, about less than 2.0%, about less than 2.5%, about less than 5%, about less than 7.5%, about less than 10%, about less than 12.5%, or greater. It will be understood that DMSO may contain one or more other impurities in addition to water.

In an embodiment, a composition comprises an iodophor, a penetrant, and further comprises water. In an embodiment, a composition comprises an anyhydrous iodophor and/or an anyhydrous penetrant, and further comprises water. In an embodiment, a composition comprises PVP-I, DMSO, and further comprises water. In an embodiment, a composition comprises an iodophor and a penetrant, and further comprises water, wherein the water content is about at least 0.01%, about at least 0.02%, about at least 0.03%, about at least 0.04%, about at least 0.05%, about at least 0.06%, about at least 0.07%, about at least 0.08%, about at least 0.09%, about at least 0.1%, about at least 0.2%, about at least 0.3%, about at least 0.4%, about at least 0.5%, about at least 0.6%, about at least 0.7%, about at least 0.8%, about at least 0.9%, about at least 1.0%, about at least 1.5%, about at least 2.0%, about at least 2.5%, about at least 5%, about at least 7.5%, about at least 10%, about at least 12.5%, or greater. In an embodiment, a composition comprises an iodophor and a penetrant, and further comprises water, wherein the water content is about less than 0.01%, about less than 0.02%, about less than 0.03%, about less than 0.04%, about less than 0.05%, about less than 0.06%, about less than 0.07%, about less than 0.08%, about less than 0.09%, about less than 0.1%, about less than 0.2%, about less than 0.3%, about less than 0.4%, about less than 0.5%, about less than 0.6%, about less than 0.7%, about less than 0.8%, about less than 0.9%, about less than 1.0%, about less than 1.5%, about less than 2.0%, about less than 2.5%, about less than 5%, about less than 7.5%, about less than 10%, about less than 12.5%, or greater. In an embodiment, a composition comprises an iodophor and a penetrant, and further comprises water, wherein the water content is about 0.01% to about 12.5%, about 0.02% to about 10.0%, about 0.03% to about 7.5%, about 0.04% to about 5%, about 0.05% to about 2.5%, about 0.06% to about 2%, about 0.07% to about 1.5%, about 0.08% to about 1%, about 0.09% to about 0.9%, about 0.1% to about 0.8%, or about 0.2% to about 0.7%. In an aspect, the water may be derived from a component of the composition. In another aspect, the water may be specifically added to the composition.

In an embodiment, a composition comprises at least one of United States Pharmacopeial Convention (USP) grade DMSO, Active Pharmaceutical Ingredient (API) grade DMSO, analytical grade DMSO, and American Chemical Society (ACS) Spectrophotometric grade DMSO. In an embodiment, a composition comprises DMSO having <0.1% water by KF titration and >99.9% determined on an anyhdrous basis.

As set forth above, the percent amount of DMSO in a composition is described in a weight-to-weight (w/w) ratio with respect to one or more other components of the composition, unless otherwise indicated. In an embodiment, the weight percent DMSO is the balance of the weight percent after addition of PVP-I. By way of a non-limiting example, a composition may comprise 1 weight percent (1%) PVP-1 and 99 weight percent (99%) DMSO. It will be understood that in the foregoing example, the DMSO component of the composition may be completely anhydrous, substantially anhydrous, or may contain water to a measurable degree. In an embodiment, the weight percent DMSO is the balance of the weight percent after addition of PVP-I and any other components (e.g., co-solvent, water, additional active ingredient, etc. . . .). In an embodiment, the weight percent DMSO is the balance of the weight percent after addition of iodophor and other components, if any. In an embodiment, the weight percent penetrant in a composition is the balance of the weight percent after addition of iodophor and other components, if any.

In an embodiment, a composition comprises DMSO in the range of 50% to 99.99%. In an embodiment, a composition comprises DMSO in the range of 1% to 99.99%. In another embodiment, a composition comprises DMSO in the range of 5% and 99.9%. In another embodiment, a composition comprises DMSO in the range of 10% and 99.9%. In another embodiment, a composition comprises DMSO in the range of 20% and 99.9%. In another embodiment, a composition comprises DMSO in the range of 30% and 99.9%. In another embodiment, a composition comprises DMSO in the range of 40% and 99.9%. In another embodiment, a composition comprises DMSO in the range of 50% and 99.9%. In another embodiment, a composition comprises DMSO in the range of 60% and 99.9%. In another embodiment, a composition comprises DMSO in the range of 70% and 99.9%. In another embodiment, a composition comprises DMSO in the range of 80% and 99.9%, and in yet another embodiment, between 90% and 99.9%. In an embodiment, a composition comprises DMSO in weight percent of about at least 80%, about at least 85%, about at least 87.5%, about at least 90%, about at least 91%, about at least 92%, about at least 93%, about at least 94%, about at least 95%, about at least 96%, about at least 97%, about at least 98%, about at least 99%, or about at least 99.9%.

In an embodiment, a composition comprises DMSO but does not comprise any additional solvent (e.g., co-solvent) or penetrant. In another embodiment, a composition comprises DMSO in the range of about 0.01% to 99.99% and further comprises at least one co-solvent in the range of 0.01% to about 99.99%. In an embodiment, a composition comprises DMSO and further comprises at least one co-solvent in the range of about 0.1% to about 50%.

In another embodiment, a composition comprises DMSO and further comprises at least one co-solvent in the range between about 5% and about 50%. In another embodiment, a composition comprises DMSO and further comprises at least one co-solvent in the range between about 10% and about 99%. In another embodiment, a composition comprises DMSO and further comprises at least one co-solvent in the range between about 20% and about 95%. In an embodiment, a composition comprises DMSO and further comprises at least one co-solvent in the range of about 50% to about 60%, about 60% to about 80%, about 70% to about 90%, and about 80% to about 95%. In an aspect, water is a co-solvent. In an embodiment, a composition comprises DMSO, water, and at least one additional co-solvent.

In an embodiment, a composition comprises DMSO in the range of about 0.01% to 99.99% and further comprises at least one penetrant in the range of 0.01% to about 99.99%. In an embodiment, a composition comprises DMSO and further comprises at least one penetrant in the range of about 0.1% to about 50%. In another embodiment, a composition comprises DMSO and further comprises at least one penetrant in the range between about 5% and about 50%. In another embodiment, a composition comprises DMSO and further comprises at least one penetrant in the range between about 10% and about 99%. In an embodiment, a composition comprises DMSO, at least one co-solvent, and at least one penetrant. In an embodiment, a co-solvent is also a penetrant.

In an embodiment, a composition includes at least one antifungal agent selected from the group consisting of tolnaftate, terbinafine, undecylenic acid, clioquinol, miconazole, miconazole nitrate, clorrinazole, tioconazole, nystatin, terconazoic, butoconazole nitrate, ciclopirox olamine, econazole nitrate, triacetin, flucytosine, haloprogin, and ketoconazole. In an embodiment, the at least one antifungal agent is present of about 0.01%, about 0.05%, about 0.1%, about 0.2%, about 0.3%, about 0.4%, about 0.5%, about 0.6%, about 0.7%, about 0.8%, about 0.9%, about 1.0%, about 1.25%, about 1.50%, about 1.75%, about 2.0%, about 2.5%, about 5%, about 7.5%, about 10%, about 12.5, about 15.0%, about 20%, about 25%, about 30%, about 40%, or about 50%. In an embodiment, a composition comprises the at least one antifungal agent of 0.01%, 0.05%, 0.1%, 0.2%, 0.3%, 0.4%, 0.5%, 0.6%, 0.7%, 0.8%, 0.9%, 1.0%, 1.25%, 1.5%, 1.75%, 2.0%, 2.25%, 2.5%, 5%, 7.5%, 10.0%, 12.5%, 15%, 20%, 25%, 30%, 40%, or 50%. In another embodiment, a composition comprises the at least one antifungal agent of about 0.1% or less, about 0.5% or less, about 1% or less, about 2% or less, about 3% or less, about 4% or less, about 5% or less, about 6% or less, about 7% or less, about 8% or less, about 9% or less, about 10% or less, about 20% or less, about 25% or less, about 30% or less, about 40% or less, or about 50% or less. In another embodiment, a composition comprises the at least one antifungal agent of about 0.1% or more, about 0.5% or more, about 1% or more, about 2% or more, about 3% or more, about 4% or more, about 5% or more, about 6% or more, about 7% or more, about 8% or more, about 9% or more, about 10% or more, about 20% or more, about 25% or more, about 30% or more, about 40% or more, or about 50% or more. In another embodiment, a composition comprises the at least one antifungal agent of 0.01%, 0.05%, 0.1%, 0.2%, 0.3%, 0.4%, 0.5%, 0.6%, 0.7%, 0.8%, 0.9%, 1.0%, 2.0%, 3.0%, 4.0%, 5.0%, 6.0%, 7.0%, 8.0%, 9.0%, 10.0%, 20%, 25%, 30%, 40%, or 50%.

In an embodiment, stable preparations of at least one topical anti-fungal ingredient known in the art including, by way of non-limiting example, 1% tolnaftate, 10-25% undecylenic acid, clioquinol 3% and/or miconazole nitrate 2%, can be prepared in DMSO solvent systems where PVP-I is also incorporated as a long-term preservative. These solutions demonstrate remarkable long-term stability where both the PVP-I component and the anti-fungal component are able to be maintained at or above 90% of the initial concentrations. It is surprisingly found that no appreciable reaction occurs between the antifungal agent and the PVP-I. These formulations also demonstrate remarkable in vitro and in vivo efficacy as antifungal agents.

In an embodiment, where possible, compositions may include pharmaceutically acceptable salts of compounds in the composition. In an embodiment, compositions comprise acid addition salts of the present compounds. In an embodiment, compositions comprise base addition salts of the present compounds. As used herein, the term pharmaceutically acceptable salts or complexes refers to salts or complexes (e.g., solvates, polymorphs) that retain the desired biological activity of the parent compound and exhibit minimal, if any, undesired toxicological effects.

In compositions for topical administration, the mixtures are preferably formulated as aqueous solutions at a pH of 2.0 to 6.5. Preferably, the pH is adjusted to between 2.5 and 4.5. This pH range may be achieved by the inclusion of suitable acids/bases in the composition.

In an aspect, a composition may comprise one or more of an excipient, an antimicrobial agent, a preservative, a cosolvent, a surfactant, a viscosity agent, and/or a bioadhesive agent, as set forth in detail elsewhere herein.

In an aspect, a pharmaceutical preparation is a partially-alcoholic preparation. As will be understood by the skilled artisan, inclusion of a percentage of alcohol in the preparation will aid in the solubility of the components, including the PVP-I. The alcohol component will also serve as a dehydrating component for the surface to which the preparation is applied. Alcohols useful in the invention include methanol, ethanol, and isopropanol, among others.

### Lubricants

In an embodiment, a composition may comprise one or more lubricants including, but are not limited to, propylene glycol, glycerin, polyethylene glycol, dextran, blended polyvinyl alcohols, polyvinyl alcohol, polyethylene glycol, light mineral oil, hydroxypropyl methylcellulose, hypromellose, carbopol, carbomer 940 (polyacrylic acid), polyvinyl pyrrolidone, white petrolatum, soy lecithin, and sodium carboxyl methylcellulose, as well as other agents known to those skilled in the art, or any combination thereof. Typically, such lubricants are employed at a level of from 0.1% to 2% by weight. In an embodiment, the lubricants are 1.0% Propylene glycol, 0.3% glycerin, 2.7% blended polyvinyl alcohols, 1% polyvinyl alcohol, 1% polyethylene glycol, light mineral oil, 0.3% hydroxypropyl methylcellulose, 1.0% soy lecithin, 0.25% or 0.5% sodium carboxyl methylcellulose. In another embodiment, the total weight of a PVP-I, artificial-tear based lubricant is between 0.1% and 4.5%.

### Additional Antimicrobial Agents and Antibiotics

Suitable antibiotic/antimicrobial agents include, but are not limited to, fluoroquinolones (ciprofloxacin, levofloxacin, ofloxacin, moxifloxacin, gatifloxacin, and the like); Aminoglycosides (tobramycin, gentamicin, neomycin, and the like); Polymyxin B Combinations (polymyxin B/trimethoprim, Polysporin polymyxin B/bacitracin Neosporin polymyxin B/neomycin/gramicidin, and the like) and other antibiotics (azithromycin, ilotycin, erythromycin, bacitracin, and the like).

### Topical Anesthetics

Suitable topical anesthetics for the compositions and methods of the invention include, but are not limited to, lidocaine, tetracaine or a derivative or combination thereof.

### Anti-Allergic Components

Anti-allergic components include, but are not limited to, epinastine, emedastine difumarate azelastine hydrochloride, olopatadine hydrochloride, olopatadine, ketotifen fumarate, pemirolast potassium, nedocromil, lodoxamide, cromolyn and cromolyn salts, as well as zinc acetate.

### Preservatives

Preservative agents include benzalkonium chloride, thimerosal, chlorobutanol, methyl paraben, propyl paraben, phenylethyl alcohol, EDTA, sorbic acid, Onamer M, other agents known to those skilled in the art, or a combination thereof. Typically, such preservatives are employed at a level of from 0.001% to 1.0% by weight of final composition.

### Co-Solvents

The compositions of the invention may contain one or more optional co-solvents. The solubility of the components of the present compositions may be enhanced by a surfactant or other appropriate co-solvent in the composition. Such cosolvents/surfactants include polysorbate 20, 60, and 80, polyoxyethylene/polyoxypropylene surfactants (e.g. Pluronic F-68, F-84 and P-103), cyclodextrin, tyloxapol, other agents known to those skilled in the art, or a combination thereof Typically, such co-solvents are employed at a level of from 0.01% to 2% by weight of the final composition.

### Soothing Agents

Furthermore, the compositions may comprise an effective amount of a chemical agent to provide a cooling sensation to relieve mild otic irritation, enhance comfort, provide a refreshing effect, and improved sensation, when the PVP-I solution is applied to the ear. Such an agent encompasses various chemicals and chemical classes, including, but are not limited to, cooling agents such as menthol, menthol derivatives including methone glycerin acetyl and menthyl esters, carboxamides, menthane glycerol ketals, alkyl substituted ureas, sulfonamides, terpene analogs, furanones, and phosphine oxides; or camphor, and borneol.

### Viscosity Agents

The compositions of the invention may contain an optional viscosity agent—that is, an agent that can increase viscosity. Viscosity increased above that of simple aqueous solutions may be desirable to increase otic absorption of the active compound, to decrease variability in dispensing the formulation, to decrease physical separation of components of a suspension or emulsion of the formulation and/or to otherwise improve the otic formulation. Such viscosity builder agents include as examples polyvinyl alcohol, polyvinylpyrrolidone, methyl cellulose, hydroxypropylmethylcellulose, hydroxyethylcellulose, carboxymethylcellulose, hydroxypropylcellulose, other agents known to those skilled in the art, or a combination thereof. Such agents are typically employed at a level of from 0.01% to 2% by weight of the final composition.

### Bioadhesive Agents

Bioadhesive agents can be used in the compositions to increase the retention time of the drug gradient over the biological substrates. The bioadhesive agents may include but are not limited to: polyvinylpyrrolidone (PVP), xanthan gum, locust bean gum, acacia gum, hydroxypropyl methylcellulose (HPMC), sodium alginate, pectin, gelatin, carbomer, polyvinylalcohol, gellan gum, tragacanth, acacia, and sodium carboxymethyl cellulose.

### Formulations and Evaluation of Effectiveness

In an embodiment, methods and compositions of the invention can reduce the progression of infectious otitis externa such that no additional progression is detected. Any method can be used to determine whether or not the severity of a symptom or the progression rate of otitis externa is reduced. For example, a human having otitis externa can be questioned regarding pain or discomfort before and after treatment to determine whether a symptom of otitis externa (e.g., ear pain or ear itching) is reduced. In some cases, a mammal can be observed or tested for the severity of a symptom of otitis externa (e.g., ear discharge, sensitivity of the ear to pressure, sensitivity of the earlobe to touch, or reduced hearing) before and after treatment with a composition according to the instant invention to determine whether or not the severity of a symptom is reduced. In some cases, an otolaryngologist can assess the severity of otitis externa (e.g., by performing a physical examination and assigning a Grade 1 to 4 score, the characteristics of which are known in the art) before and after treatment to determine whether the severity of a symptom is reduced. To determine whether or not progression of otitis externa is reduced, a physical examination can be performed at different time points to determine the amount of erythema and/or edema in and around the ear canal. The amount of erythema and edema observed at different time points can be compared to assess the progression rate. After treatment as described herein, the progression rate can be determined again over another time interval to determine whether or not the progression rate has decreased.

Therefore, it will be understood that an effective amount of a composition of the subject invention, comprising PVP-I and DMSO is any amount that reduces the severity of a symptom or the progression of otitis externa without producing significant toxicity to the mammal For example, an effective amount of PVP-I can be from about 0.1% to about 10% (e.g., about 2%) povidone-iodine in an otic drop formulation. In an embodiment, an effective amount of a composition comprising PVP-I and DMSO can be from about 2 drops to about 8 drops of an otic drop formulation containing about 1%-2% povidone-iodine and about 30% or higher DMSO applied to the ear.

If a mammal does not appear respond to a particular amount of a composition of the invention, then the amount of one or more of the PVP-I and the DMSO, for example, can be increased. After receiving this higher concentration, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, immunocompetency of the mammal, and severity of the otitis externa may require an increase or decrease in the actual effective amount administered. The frequency of administration can be any frequency that reduces the severity of a symptom or progression rate of otitis externa without producing significant toxicity to the mammal For example, the frequency of administration can be from about once daily to about four times daily (e.g., about twice daily). The frequency of administration can remain constant or can be variable during the duration of treatment.

In another aspect, a course of treatment with a composition of the subject invention can include rest periods. For example, the composition can be administered over a one or two week period followed by a one or two week rest period, and such a regimen can be repeated multiple times. As with the effective amount, various factors can influence the actual frequency of administration used for a particular application. For example, the effective amount, duration of treatment, use of multiple treatment agents, route of administration, immunocompetency of the mammal, and severity of the otitis externa may require an increase or decrease in administration frequency. An effective duration for administering a composition provided herein can be any duration that reduces the severity of a symptom or the progression rate of otitis externa without producing significant toxicity to the mammal Thus, the effective duration can vary from several days to several weeks, months, or years. In general, the effective duration for the treatment of otitis externa can range in duration from several days to several weeks. In some cases, an effective duration can be for as long as an individual mammal is alive. Multiple factors can influence the actual effective duration used for a particular treatment. For example, an effective duration can vary with the frequency of administration, effective amount, use of multiple treatment agents, route of administration, immunocompetency of the mammal, and severity of the otitis externa.

The above diagnosis and treatment considerations can be applied in a similar manner to the treatment of otitis media and otitis externa.

### Methods of Preparation and Use

It is known to one of skill in the art that PVP-I aqueous solutions are difficult to stabilize at low PVP-I concentrations over a long period of time. By way of a non-limiting example, at concentrations of PVP-I less than about 0.7% (w/w, aqueous), PVP-I aqueous solutions rapidly decay to yield complex mixtures of iodinated and iodine-free constituents. As described herein, it was surprisingly found that in the aprotic DMSO solvent system encompassed by the disclosure set forth herein, PVP-I solutions as low as 0.1% can be easily prepared and maintained as stable compositions for long periods of time. Also as described herein, hydrated DMSO solutions prepared from aqueous PVP-I demonstrate increased stability is noted for the PVP-I component.

In an embodiment, a composition comprises dry, solid or powdered PVP-I dissolved or suspended in a composition comprising or consisting of DMSO. In another embodiment, DMSO is added to an aqueous preparation comprising or consisting of PVP-I. Based on the disclosure herein, one of skill in the art will understand how to prepare a composition to arrive at the desired amounts of iodine, iodophor, and DMSO, among other possible components of the compositions encompassed herein.

By way of a non-limiting example, a therapeutically-effective pharmaceutical composition is prepared using solid PVP-I, which is dissolved or suspended in DMSO. In an aspect, the composition is anhydrous. In an aspect, the composition is substantially anhydrous. In another embodiment, DMSO can be added to aqueous solutions of PVP-I to prepare a therapeutically-effective pharmaceutical composition. In an embodiment, DMSO is used in the range of 50%-99% as a co-solvent with water. In an embodiment, a formulation includes one or more excipients. By way of a non-limiting example, excipients include, but are not limited to, sodium chloride, sodium dihydrogen phosphate monohydrate, disodium hydrogen phosphate anhydrous and water, as well as others known to those skilled in the art.

In an embodiment, a composition is prepared by adding 10% PVP-I (w/v, aqueous) to pure DMSO q.s. to yield a resulting solution of 1% PVP-I (w/w) with DMSO. In another embodiment, compositions are prepared by dissolving solid PVP-I in pure DMSO q.s to obtain any of 0.1%, 0.2%, 0.3%, 0.4%, 0.5%, 1.0%, 1.25%, 1.5%, 2.0%, or 2.5% PVP-I (w/w), as well as about 0.1%, about 0.2%, about 0.3%, about 0.4%, about 0.5%, about 1.0%, about 1.25%, about 1.5%, about 2.0%, or about 2.5% PVP-I (w/w) compositions, with DMSO as the solvent. In yet another embodiment, compositions are prepared by dissolving solid PVP-I in pure DMSO q.s to obtain any composition set forth, described, and/or encompassed herein. Similar compositions comprising aqueous PVP-I (with and without excipients commonly used and/or known in the art) and DMSO can be prepared from a stock 10% PVP-I aqueous solution and pure DMSO. It will be understood by the skilled artisan, however, that any starting composition of PVP-I, solid or liquid, may be used when the appropriate dilutions and adjustments are made to result in the desired final PVP-I concentration. Similarly, any starting composition of iodophor or elemental iodine may be used when the appropriate dilutions and adjustments are made to result in the desired final iodophor or elemental iodine concentration, respectively.

In an embodiment, it is particularly useful for the case of otitiss that stable, anhydrous compositions that contain between 0.01%-10% PVP-I can be prepared in pure USP grade DMSO solvents.

It will be understood, based on the disclosure set forth herein, in view of the skill in the art, that specific dosage for compounds and compositions encompassed herein may be determined empirically through clinical and/or pharmacokinetic experimentation, and that such dosages may be adjusted according to prespecified effectiveness and/or toxicity criteria. It will also be understood that a specific dosage and treatment regimen for any particular patient will depend upon a variety of factors, including the activity of the specific compounds employed, the characteristics of the patient, drug combination, the judgment of the treating physician and the nature and severity of the particular disease or condition being treated.

In an embodiment, a composition set forth, described, and/or encompassed herein is useful for treating one or more of—but not limited to—onychomycosis, paronychia, verrucous warts, molluscum contagiosum, non-genital herpes simplex, scars, gram negative toe-web infection, psoriatic nail dystrophy, and tinea pedis. In an embodiment, the composition comprises PVP-I and DMSO. In an embodiment, the composition consists essentially of PVP-I and DMSO. In an embodiment, the composition consists of PVP-I and DMSO.

In an embodiment, a therapeutic composition is prepared by optimizing one or more compounds for use in a dosage form different than that which is typically used for the compound. In an embodiment, a compound that is not typically administered in a topical dosage form is developed for use in a topical dosage form. The chemical and biological assays required for such development are known to one of skill in the art. The disclosure herein provides the skilled artisan with the guidance as to how to prepare such compounds and compositions comprising such compounds.

In an embodiment, a method of treating a subject having an otitis includes administration of a composition set forth, described, and/or encompassed herein to treat the otitis, and the treatment of the otitis includes at least one of preventing or slowing the progression of the infection, preventing the spread of the infection, eradicating at least some of the infection, and eradicating the entire infection.

In an embodiment, a therapeutic composition is administered on a schedule once a day. In an embodiment, a therapeutic composition is administered twice a day. In an embodiment, a therapeutic composition is administered three times a day, four times a day, five times a day, or more. In an embodiment, a therapeutic composition is administered less frequently than once a day. In an embodiment, a therapeutic composition is administered once every two days, once every three days, once every four days, once every five days, once every six days, or once every seven days. In an embodiment, a therapeutic composition is administered less frequently than once a week. In an embodiment, a therapeutic composition is administered once a month. In an embodiment, a therapeutic composition is administered twice a month.

In an embodiment, a therapeutic dosing regimen is continued for at least one day, at least two days, at least three days, at least four days, at least five days, at least six days, or at least seven days. In an embodiment, a therapeutic dosing regimen is continued for at least one week, at least two weeks, at least three weeks, at least four weeks, at least six weeks, at least eight weeks, at least ten weeks, at least twelve weeks, at least fourteen weeks, or at least sixteen weeks. In an embodiment, a therapeutic dosing regimen is continued for at least one month, at least two months, at least three months, at least four months, at least five months, at least six months, at least nine months, or at least twelve months.

The invention is further described by the following examples. In an aspect, the following examples demonstrate effective and/or successful treatment of the identified conditions using compositions and methods encompassed by the present disclosure. It should be recognized that variations based on the inventive features are within the skill of the ordinary artisan, and that the scope of the invention should not be limited by the examples. To properly determine the scope of the invention, an interested party should consider the claims herein, and any equivalent thereof In addition, all citations herein are incorporated by reference, and unless otherwise expressly stated, all percentages are by weight.

### EXAMPLE 1

### Acute Otits Externa In A Child Treated With 2% Povidone-Iodine Gel

This patient was suffering from acute otitis externa. The condition is often very painful with severe irritation occurring when the child moves the head or moves. A composition as disclosed herein using 2.0% PVP-I in 44% USP Grade DMSO with water and 2% hydroxyethylcellulose (HEC) to form a thick gel. The patient was treated by applying the gel topically twice each day to the external ear canal. Within four days the infection resolved and the patient was not in pain.

### EXAMPLE 2

### Acute Otits Externa In A Child Treated With Highly Viscous 2% Povidone-Iodine Gel

This patient was suffering from acute otitis externa. The condition is often very painful with severe irritation occurring when the child moves the head or moves. A composition as disclosed herein using 2.0% PVP-I in 44% USP Grade DMSO with water and 4% hydroxyethylcellulose (HEC) to form a viscous gel having viscosity over 300,000 cps. The patient was treated by applying the gel topically once a day to the external ear canal. Within two days the infection resolved and the patient was not in pain.

It will be appreciated by those skilled in the art that changes could be made to the exemplary embodiments shown and described above without departing from the broad inventive concept thereof It is understood, therefore, that the disclosure herein is not limited to the exemplary embodiments shown and described, but it is intended to cover modifications within the spirit and scope of the present invention as defined by the claims. For example, specific features of the exemplary embodiments may or may not be part of the claimed invention and features of the disclosed embodiments may be combined. Unless specifically set forth herein, the terms “a”, “an” and “the” are not limited to one element but instead should be read as meaning “at least one”.

It is to be understood that at least some of the descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent that the method does not rely on the particular order of steps set forth herein, the particular order of the steps should not be construed as limitation on the claims.

The claims directed to the method of the present invention should not be limited to the performance of their steps in the order written, and one skilled in the art can readily appreciate that the steps may be varied and still remain within the spirit and scope of the present invention.

1. An otic composition comprising 0.1%-10% w/w povidone-iodine and 30%-99% w/w DMSO and at least one optically acceptable excipient.

2. The composition of claim 1, wherein the composition comprises a gelling agent.

3. The composition of claim 2, wherein the gelling agent is selected from hydroxyethyl cellulose, hydroxymethyl cellulose, and hydroxypropyl methylcellulse.

4. The composition of claim 1, wherein the composition comprises PEG.

5. The composition of claim 1, wherein the composition comprises petrolatum.

6. The composition of claim 1, wherein the composition is steroid-free.

7. A method for preventing or treating an otic infection or infestation, said method comprising:
applying or administering a composition of claim 1.

8. The method of claim 6, wherein the otic infection or infestation is ototis externa.

9. The method of claim 6, wherein the otic infection is otitis media.

10. The method of claim 6, wherein the otic infection is a viral infection.

11. The method of claim 6, wherein the otic infection or infestation is a bacterial infection.

12. The method of claim 6, wherein the otic infection or infestation is a fungal infection.

13. The method of claim 6, wherein the otic infection or infestation is amoebal infection or infestation.

14. The method of claim 7, wherein the composition of claim 1 is steroid-free.
